(19) United States
(12) Patent Application Publication
(10) Pub. No.: US 2022/0390539 A1
(43) Pub. Date: Dec. 8, 2022

(54) MAGNETIC RESONANCE IMAGING APPARATUS AND IMAGE PROCESSING APPARATUS

(71) Applicant: FUJIFILM Healthcare Corporation, Kashiwa-shi (JP)

(72) Inventors: Suguru Yokosawa, Chiba (JP); Toru Shirai, Chiba (JP)

(21) Appl. No.: 17/828,193

(22) Filed: May 31, 2022

(30) Foreign Application Priority Data
Jun. 3, 2021 (JP) 2021-093814

(51) Int. Cl. G01R 33/56; G01R 33/565; G01R 33/54; G06T 7/246; G06T 7/00

(52) U.S. Cl. CPC G01R 33/5608; G01R 33/56509; G01R 33/543; G06T 7/248; G06T 7/0012; G06T 2207/10088; G06T 2207/30004; G06T 2207/10016; G06T 2200/24

(57) ABSTRACT

The present invention is to acquire a multiphase image while avoiding extension of imaging time and excluding an influence of displacement of an image of each multiphase due to a motion. A method for collecting measurement data is to repeat sampling such that low-frequency data and high-frequency data have different densities. At this time, a sampling interval is set shorter than a motion cycle. Motion information is acquired in parallel with imaging, and measurement data obtained in time series is divided into a plurality of time phases based on the motion information so as to obtain a multiphase image. Displacement correction between multiphase images is performed, and then the multiphase images are integrated. Alternatively, measurement data after the displacement correction is used to generate a time-series image.

### BACKGROUND OF THE INVENTION

### 1. Field of the Invention

The present invention relates to a magnetic resonance imaging (hereinafter, referred to as MRI) apparatus, and in particular to processing of measurement data imaged in existence of a motion.

### 2. Related Art

In MRI imaging, an artifact occurs in an image when a target moves during imaging. Therefore, particularly when imaging a body trunk, which is affected by respiratory motions or heartbeats, an imaging method that eliminates influences of motions, such as breath-hold imaging or respiratory gated imaging, is used. The breath-hold imaging imposes a burden on a patient, and time is limited for the patient to hold the breath, which causes limitation on imaging conditions, for example, imaging needs to be performed with reduced resolution. The respiratory gated imaging generates an image based on measurement data acquired at a specified segment of the respiratory cycle, and thus requires imaging over a plurality of cycles, which causes a problem of extended measurement time.

Japanese Patent No. 6682243 discloses a technique for an imaging apparatus such as an X-ray CT apparatus or an MRI apparatus. The technique includes: calculating displacement of a target portion due to a motion based on a morphological image obtained at each time phase of the motion; and correcting an influence of the motion in functional images collected apart from the morphological images based on the calculated displacement by a nuclear medicine imaging apparatus such as a PET apparatus. This is a method for correcting influence of a motion in functional images acquired by a nuclear medicine imaging apparatus, which has a longer imaging time than the X-ray CT apparatus or the MRI apparatus. In order to solve the problem of the breath-hold imaging or the gated imaging in the MRI apparatus described above by the method based on Japanese Patent No. 6682243, this technique cannot be directly used to perform motion correction, unless combined with a morphological image data collection apparatus having an even shorter imaging time than the MRI apparatus.

In order to solve such a problem unique to the MRI apparatus, JP-A-2019-130307 discloses a technique of generating intermediate data based on each of a plurality of pieces of primary measurement data obtained by under-sampling over a plurality of time phases, generating secondary measurement data corresponding to full sampling by inversely transforming the intermediate data, and generating image data of each cardiac phase by combining data of a peripheral region obtained at the same cardiac phase as a region near a center of the primary measurement data among data of the peripheral region included in the secondary measurement data and reconstructing an image, so as to appropriately capture an image for an imaging target in motion.

### SUMMARY OF THE INVENTION

The technique disclosed in JP-A-2019-130307 is a technique of acquiring a multiphase image without being affected by a difference in a collection time of measurement data included in a certain segment of a motion cycle with respect to imaging of a portion involving a cyclic motion by division of sampling and combination processing of sampling data. However, the limitation due to the gated imaging still exists. In addition, improvement of image quality of images of each time phase is limited.

An object of the invention is to provide an MRI apparatus that can present an image of any time phase that is a high-quality multiphase image while excluding an influence of a motion.

In order to solve the above problem, the MRI apparatus according to the invention adopts a method for collecting measurement data that is a collecting method of repeating sampling such that low-frequency data and high-frequency data have different densities, performing displacement correction between time phase images for each time phase image obtained by dividing measurement data obtained in time series into each time phase, and then integrating the time phase images. At this time, it is possible to obtain a high-quality integrated image of any time phase, by using all measurement data and converting different time phase images in accordance with a reference of displacement correction. Alternatively, data after the displacement correction rearranged in a measurement order is used to generate a time-series image.

That is, the MRI apparatus according to the invention includes: a measurement unit configured to measure an echo signal generated by nuclear magnetic resonance from a subject and collect measurement data arranged in k-space; an image processing unit configured to generate an image by processing the measurement data collected by the measurement unit; and a control unit configured to control the measurement unit such that the measurement unit repeats sampling of measurement data including low-frequency data and high-frequency data such that the low-frequency data has a sampling density in the k-space higher than a sampling density of the high-frequency data. The image processing unit includes: a motion information input unit configured to receive motion information of the subject; a multiphase image generation unit configured to use the motion information to divide the measurement data into measurement data at a plurality of segments of a motion cycle of the subject, and generate a multiphase image based on the divided measurement data; a displacement amount calculation unit configured to calculate a displacement amount between each phase of the multiphase image due to the motion for the multiphase image; a displacement correction unit configured to use the displacement amount to generate displacement-corrected data from data related to each phase of the multiphase image; and a collection period reference image generation unit configured to generate an image using the displacement-corrected data corresponding to the measurement data collected in any collection period by the measurement unit. The image generated by the collection period reference image generation unit includes, for example, an integrated image in which each of the multiphase image after correction are integrated, and a time-series image generated by rearranging the displacement-corrected data after correction in a measurement order.

An image processing apparatus according to the invention is an apparatus independent of the MRI apparatus, and functions as an image processing unit of the MRI apparatus as described above.

According to the invention, it is possible to obtain an image of each time phase while excluding an influence of a motion.

### BRIEF DESCRIPTION OF THE DRAWINGS

FIG. 1 is a diagram illustrating an overview of an MRI apparatus.

FIG. 2 is a diagram illustrating a configuration of an image processing unit.

FIG. 3 is a diagram illustrating a relationship between an image processing apparatus and the MRI apparatus.

FIG. 4 is a flowchart illustrating a flow of processing performed by the image processing unit.

FIG. 5 is a diagram illustrating an example of sampling performed by a measurement unit according to a first embodiment.

FIG. 6 is a diagram illustrating a process performed by the image processing unit according to the first embodiment.

FIGS. 7A to 7C are diagrams illustrating an example of sampling performed by the measurement unit according to the first embodiment (modification).

FIG. 8 is a diagram illustrating a configuration of an image processing unit according to a second embodiment.

FIG. 9 is a diagram illustrating an example of a GUI for receiving reconstruction conditions according to the second embodiment.

FIG. 10 is a diagram illustrating a configuration of an image processing unit according to a third embodiment.

FIG. 11 is a flowchart illustrating a flow of processing performed by the image processing unit according to the third embodiment.

FIG. 12 is a diagram illustrating a process performed by the image processing unit according to the third embodiment.

FIG. 13 is a diagram illustrating a configuration of an image processing unit according to a fourth embodiment.

FIG. 14 is a diagram illustrating an example of a GUI for receiving reconstruction conditions according to the fourth embodiment.

### DESCRIPTION OF EMBODIMENTS

Hereinafter, embodiments of an MRI apparatus according to the invention will be described with reference to the drawings.

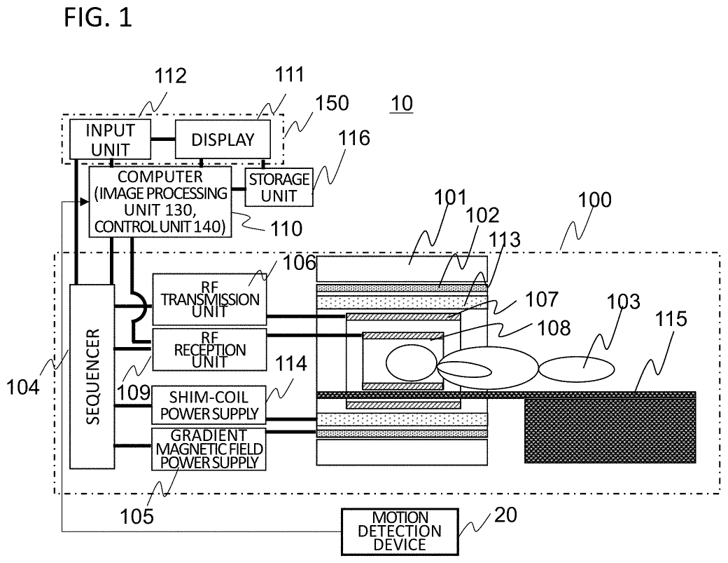

FIG. 1 is a diagram illustrating an overall configuration of an MRI apparatus applied with the invention. Similarly to a general MRI apparatus, an MRI apparatus 10 includes: a measurement unit 100 that applies a high-frequency magnetic field to a subject 103 arranged in a static magnetic field space to measure a nuclear magnetic resonance signal induced in atomic nucleuses of atoms constituting a tissue of the subject; an image processing unit 130 that uses the nuclear magnetic resonance signal to reconstruct an image of the subject 103; and a control unit 140 that controls the entire apparatus including the measurement unit 100 and the image processing unit 130.

The measurement unit 100 includes a static magnetic field magnet (static magnetic field generation unit) 101, a gradient magnetic field generation unit (102, 105), an RF transmission unit 106 that generates a high-frequency pulse (hereinafter referred to as an RF pulse), an RF reception unit 109 that receives the NMR signal generated from the subject 103, and a bed device 115 for arranging the subject 103 in the static magnetic field space generated by the static magnetic field magnet 101.

The gradient magnetic field generation unit includes three sets of gradient magnetic field coils 102 that generate gradient magnetic fields in three axial directions orthogonal to each other, and a gradient magnetic field power supply 105 that drives the gradient magnetic field coils. The gradient magnetic field generation unit controls a drive current to be applied to each gradient magnetic field coil, so as to apply a magnetic field gradient in any direction (a phase encoding direction, a readout direction, a slice direction, or the like) with respect to the static magnetic field formed by the static magnetic field magnet 101. Thus, the NMR signal can be encoded and given position information. The example shown in FIG. 1 is further provided with a shim coil 113 for increasing uniformity of the static magnetic field and a power supply 114 for driving the shim coil 113.

The RF transmission unit 106 includes an RF transmission coil 107 that emits an RF pulse, and supplies a high-frequency signal to the RF transmission coil 107. The RF reception unit 109 is connected to an RF reception coil 108 disposed close to the subject 103, and performs amplification, quadrature detection, A/D conversion, and the like on the NMR signal detected by the RF reception coil 108. The NMR signal is usually measured as an echo signal using inversion of a readout gradient magnetic field pulse, and thus is hereinafter referred to as an echo signal. The echo signal is sampled within a predetermined sampling time and becomes time-series digital data.

The measurement unit 100 further includes a measurement control unit (sequencer) 104 that controls operations of the gradient magnetic field power supply 105, the RF transmission unit 106, and the RF reception unit 109 based on a predetermined pulse sequence. The measurement control unit 104 operates under control of the control unit 140, and performs control such that measurement data necessary for image reconstruction is collected in a predetermined sampling order in accordance with the predetermined pulse sequence.

The image processing unit 130 and the control unit 140 can be built in a machine including a CPU and a memory, and these functions are implemented in a computer 110 in the example shown in FIG. 1. The computer 110 includes an input device 112 for receiving an input from a user, a storage device 116 for storing an image, a processing result of a calculation unit, and the like, and a display 111 for presenting the image, the processing result, and the like to the user. The display 111 and the input device 112 are usually disposed close to each other, and function as a user interface (UI) unit 150 for the user to interactively communicate with the MRI apparatus 10.

The image processing unit 130 uses the NMR signal to reconstruct an image, performs calculation and processing on measurement data and image data, and performs division and integration of images, correction of images, reduction of artifacts, and the like. When the above image processing is performed, the image processing unit 130 receives motion information from a device (motion detection device) 20 that detects a motion of the subject, and uses the motion information to perform processing for reducing an influence of the motion. The motion detection device 20 may be any known measuring instrument or detector, such as an electrocardiograph or a heart rate monitor attached to the subject, a balloon for detecting respiratory motions, and a camera for monitoring motions of the subject. The motion information obtained from the motion detection device 20 is, for example, an electrocardiogram, a heart rate diagram, or a graph indicating a motion cycle or the like. The image processing unit 130 captures the motion information from the motion detection device 20 and performs image processing in parallel with progress of imaging. Alternatively, the motion detection device 20 here may replace measurement data included in an imaging sequence with processed navigator data.

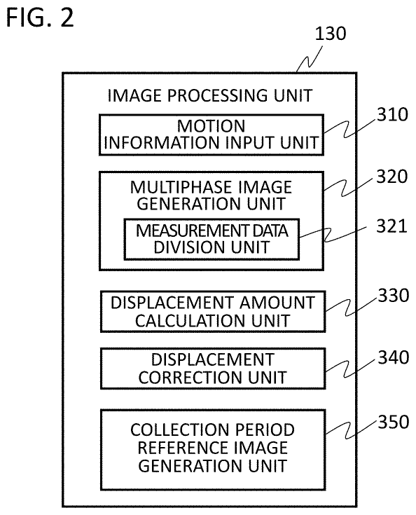

FIG. 2 shows a configuration example of the image processing unit 130 which implements such a function. The image processing unit 130 includes: a motion information input unit 310 that captures information from the motion detection device 20; a multiphase image generation unit 320 that uses the motion information captured by the motion information input unit 310 to divide the measurement data into measurement data of a plurality of time phases of the motion of the subject, and generates a time phase image of each time phase based on the divided measurement data of each time phase; a displacement amount calculation unit 330 that uses the time phase image of each time phase to calculate a displacement amount due to the motion; a displacement correction unit 340 that uses the displacement amount to correct data related to the time phase image; and a collection period reference image generation unit 350 that generates an image using the corrected time phase image collected in any collection period or the measurement data thereof. In the illustrated example, the multiphase image generation unit 320 includes a measurement data division unit 321.

The control unit 140 controls operations of the measurement unit 100 through the measurement control unit 104, and performs control of causing a display unit to display an image as a processing result of the image processing unit 130, various data, a graphic user interface (GUI) for the user, and the like (a function as a display control unit). Functions of units of the image processing unit 130 and the control unit 140 are implemented, for example, by the CPU of the computer 110 executing programs corresponding to the functions. Some of the functions can also be implemented by hardware such as an application specific integrated circuit (ASIC) or a field programmable gate array (FPGA).

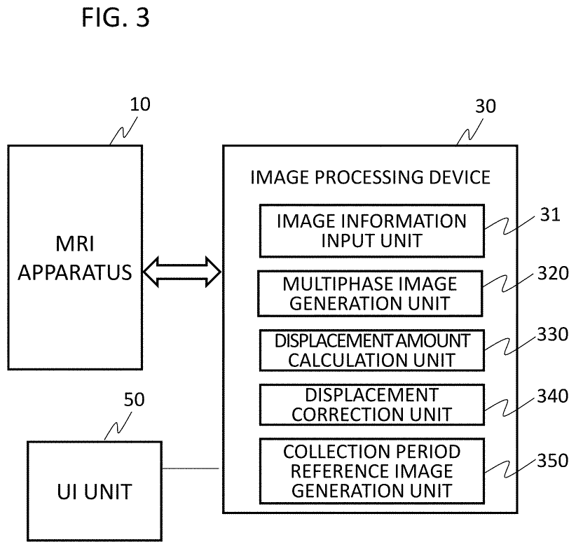

As shown in FIG. 3, the image processing unit 130 may be implemented by an image processing apparatus 30 mounted on an independent computer or workstation separate from the MRI apparatus 10. Such an image processing apparatus 30 may be connected to the MRI apparatus 10 in a wired or wireless manner, or may be connected to the MRI apparatus 10 through the Internet or the like. The image processing apparatus 30 includes an image information input unit 31 that receives measurement data and image data attached with the motion information. The image processing apparatus 30 further includes a UI unit 50, a storage device (not shown), and the like.

Hereinafter, the image processing unit 130 of the MRI apparatus 10 will be described. This description is also applicable to processing performed by the independent image processing apparatus 30.

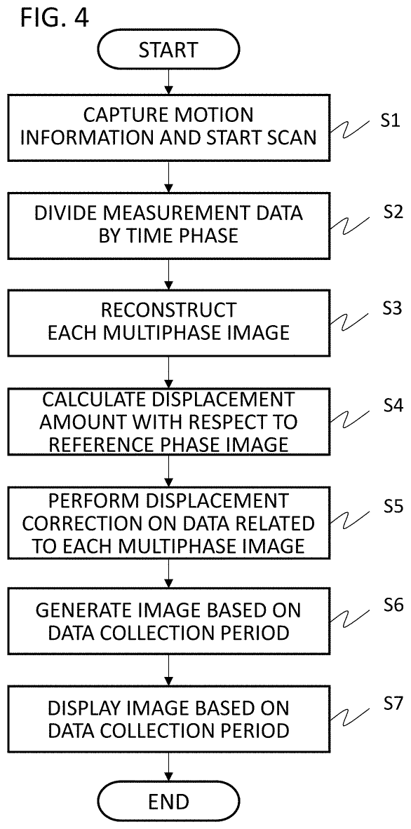

Next, based on the above configuration, a flow of imaging performed by the MRI apparatus according to the present embodiment will be described. FIG. 4 shows a flow of operations.

The sequencer 104 calculates a pulse sequence to be used for imaging (referred to as an imaging sequence) from a predetermined pulse sequence selected corresponding to imaging and imaging conditions such as imaging parameters input by the user from, for example, the UI unit 150, and performs imaging by operating the RF transmission unit, the gradient magnetic field generation unit, and the RF reception unit in accordance with the imaging sequence (S1).

The pulse sequence as a basis of the imaging sequence is not particularly limited, may be any of a spin echo sequence and a gradient echo sequence, and may be applied to either a two-dimensional sequence or a three-dimensional sequence. For example, in a case of the three-dimensional sequence, a gradient magnetic field pulse for encoding an echo signal is applied in two directions including a phase encoding direction and a slice encoding direction, and measurement of the echo signal is repeated while changing an encoding amount thereof, thereby collecting three-dimensional k-space data.

A method for scanning k-space data may be Cartesian scan in which k-space is scanned in parallel, radial scan in which the k-space is radially scanned, spiral scan in which the k-space is spirally scanned, and the like. Any of these methods can be adopted, but in any of these methods, sampling is repeated such that low-frequency data in the k-space has a sampling density higher than a sampling density of high-frequency data. The imaging parameters (TR and the like) of the pulse sequence are set such that the repetition cycle of sampling is shorter than a motion cycle. The low-frequency data refers to data in a low-frequency region of the echo signal in the vicinity of a center of the k-space data, and data close to the periphery of the k-space data or data excluding the low-frequency data is referred to as high-frequency data here. A k-space scanning method will be specifically described in subsequent embodiments.

In parallel with the imaging (S1), motion monitoring is performed by the motion detection device 20 measuring the motion of the subject 103, and the motion information input unit 310 captures the motion information output from the motion detection device 20.

The image processing unit 130 uses the k-space data obtained by the imaging to reconstruct an image. At this time, the multiphase image generation unit 320 (measurement data division unit 321) first uses the motion information captured by the motion information input unit 310 to divide the measurement data into data of each time phase of the motion (S2). For example, when one motion cycle is divided into N time phases, time-series measurement data collected over a plurality of cycles (M cycles) is divided into N×M pieces of measurement data, and measurement data of the same time phase is collected as measurement data of each of the N time phases. The measurement data of each time phase does not include all the data in the k-space. Since the low-frequency data is collected at a high sampling density in each repetition for collecting the measurement data, the collected data include the low-frequency data, which can be reconstructed to an image.

Next, the multiphase image generation unit 320 reconstructs the image based on the measurement data of each time phase (S3). Examples of an image reconstruction method include, in addition to inverse fast Fourier transform (FFT), a method using estimation calculation such as compressed sensing (CS), calculation of parallel imaging (PI), and the like, but are not limited thereto. Here, the inverse FFT, which has a small calculation load, is used.

The displacement amount calculation unit 330 uses the reconstructed time phase image to calculate a difference in position between the images as a displacement amount (displacement of another time phase with reference to a certain time phase) (S4), and the displacement correction unit 340 uses the calculated displacement amount to correct data related to the time phase image (S5). A specific method for displacement correction will be described in detail in subsequent embodiments.

Finally, the collection period reference image generation unit 350 uses the corrected data to generate an image based on a collection period while excluding the influence of the motion (S6). Here, the image generated based on a collection period means an image reconstructed using the data after displacement correction corresponding to the data measured in any collection period, which is distinguished from the image generated based on actually-measured measurement data. Such reconstructed image includes, for example, an integrated image in which the time phase images created using all the corrected data corresponding to all the measurement data are integrated, and a time-series image obtained by creating an image in each data collection period based on the corrected data rearranged in a measurement order. In either case, since a positional deviation between the time phase images caused by the motion is corrected, the generated image becomes an image in which the influence of the motion is reduced. The integrated image generated by integrating the time phase images is a high-quality image of a desired time phase. On the other hand, the time-series image is not affected by the motion cycle, and thus is a high-quality image including data of other time phases, which are not used at the time of image generation in related art. The image is stored in the storage device 116 or displayed on the display 111 as necessary (S7).

According to the present embodiment, it is possible to obtain a high-quality image while avoiding problems such as limitation due to breath holding and extension of imaging time in a case of gated imaging, and excluding the influence of the motion.

Hereinafter, a specific embodiment of processing of the MRI apparatus including the k-space scanning method will be described.

### First Embodiment

The present embodiment generates an integrated image as an image based on the data collection period. In the present embodiment as well, configurations and operations of the devices shown in FIGS. 1, 2, and 4 are similar, and thus, processing of the present embodiment will be described with reference to these drawings as appropriate in the following. However, the present embodiment will be described by replacing the collection period reference image generation unit 350 in FIG. 2 with an image integration unit 350.

In the present embodiment, for example, the radial scan is adopted as the k-space scanning method.

In the radial scan, when an echo signal is sampled by frequency encoding, the k-space is radially scanned by sampling with a combination of different magnetic field strengths of gradient magnetic fields in two directions. For example, in a case of three-dimensional imaging, frequency encoding is radially scanned in two-dimensional k-space, and an encoding gradient magnetic field pulse is used only in one direction. Alternatively, the frequency encoding is scanned in a direction parallel to one axis of the k-space, and an intensity of the gradient magnetic field pulse is changed such that the frequency encoding is radially scanned by a combination of intensities of the gradient magnetic field pulses in two directions including the phase encoding direction and the slice encoding direction. In the radial scan, the low-frequency data can be sampled at a higher frequency than the high-frequency data as a result of repeating the radial scan.

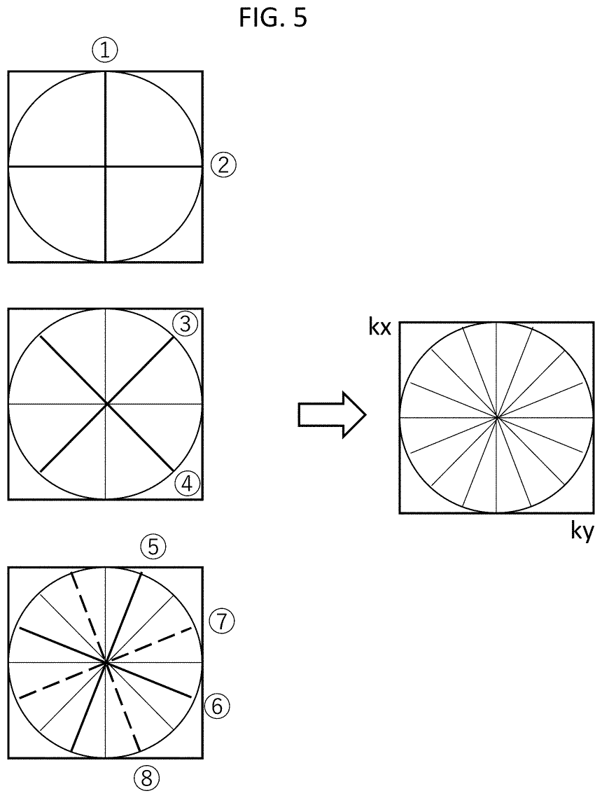

The pulse sequence may be any of the spin echo sequence and the gradient echo sequence, and an interval of sampling echo signals of each line is set to be sufficiently shorter than the motion cycle as a problem. The interval of sampling echo signals of each line means a time for collecting data of one line in k-space data in two k-space directions (kx, ky) as shown in FIG. 5, for example, in a case of three-dimensional radial scan. When the motion is a respiratory motion, one cycle is about 3 to 5 seconds and one cardiac cycle is about 1 second. Therefore, it can be said that sampling intervals are sufficiently short as long as the sampling intervals are 300 ms or less and 100 ms or less, respectively.

An order for collecting echo signals in the radial scan is set to an order in which the k-space is scanned as uniformly as possible. In the radial scan, central data is generally sampled repeatedly in the k-space, and thus the sampling density of the low-frequency data is high. However, when the collection is sequentially performed in a clockwise or counterclockwise order, the measurement data is spatially biased in the k-space until a round of k-space scanning is completed. In the present embodiment, for example, the (2n−1)-th (where n is an integer of 1 or more) and the 2n-th form an angle of 90 degrees with respect to each other, and the third and the subsequent are each a central angle between adjacent collected angles. When collection is performed in such an order, as shown in FIG. 5, radial measurement data forming an angle of 45 degrees with each other are obtained by sampling the echo signals four times, radial measurement data forming 22.5 degrees with each other are obtained by sampling the echo signals eight times, data near the center of the k-space (low-frequency data) can be scanned at a high frequency, and the k-space can be scanned uniformly. In FIG. 5, numbers enclosed by circles indicate an order for collecting data.

Such measurement of the echo signal is executed over a plurality of motion cycles to collect time-series measurement data (S1). The measurement data is time-series measurement data having information on sampling time and information on angles in the k-space.

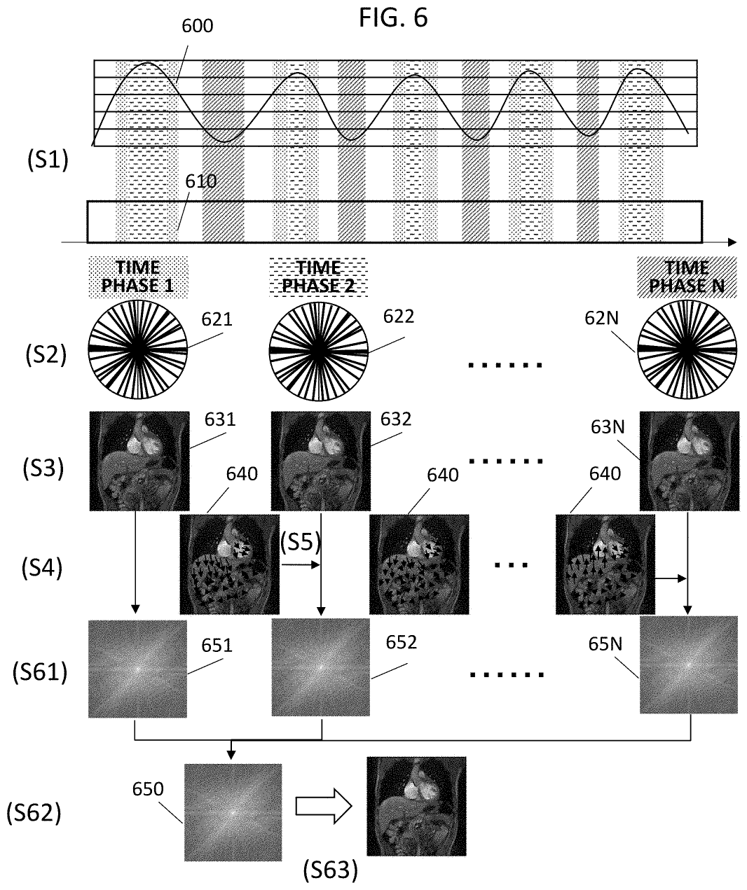

Next, the measurement data division unit 321 uses the motion information to divide the time-series measurement data into data of each time phase of the motion (S2). FIG. 6 shows an example of dividing the measurement data. In FIG. 6, processing corresponding to steps in FIG. 4 is shown together with reference signs of the steps. A graph shown in S1 of FIG. 6 is a graph 600 showing the motion cycle captured by the motion information input unit 310, and time-series measurement data 610 measured in S1 is shown below. The measurement data 610 is associated with the time phase of the motion cycle based on time information when each piece of data is acquired. In FIG. 6, three time phases 1, 2, and N are representatively shown among N time phases 1 to N of the motion cycle. The measurement data division unit 321 rearranges the measurement data obtained in the same time phase of each cycle in the k-space of each time phase as shown in (S2) of FIG. 6. For example, the measurement data obtained in the time phase 1 (data collected in five sections in FIG. 6) is defined as k-space data 621 of the phase 1, the measurement data obtained in the time phase 2 is defined as k-space data 622 of the time phase 2, and the measurement data obtained in the time phase N is defined as k-space data 62N of the time phase N. At this time, data at the same angle is added when overlapping with each other. The k-space data 621 to 62N of the time phases 1 to N obtained in this manner are sparser than the data obtained by fully scanning the k-space, but is sampled such that the low-frequency data near the center of the k-space is denser, and thus include a sufficient amount of low-frequency data and high-frequency data sparser than the low-frequency data, which enables image reconstruction.

The multiphase image generation unit 320 performs image reconstruction on the measurement data 621 to 62N for each phase to generate time phase images 631 to 63N (S3). The image reconstruction is the same as image reconstruction of normal radial scan. Examples thereof include a method of performing gridding to rearrange data in a square lattice and then converting the data into real-space data by the inverse FFT.

Next, the displacement amount calculation unit 330 calculates a deviation amount (displacement amount) 640 of positions of the time phase images 631 to 63N (S4). As to the displacement amount, one of the N time phase images is set as a reference phase image, and the displacement amount with respect to the reference phase image is calculated. The reference phase image may be, for example, an image of an initial phase among the measurement data arranged in time series, or may be an image of a time phase in which the motion changes least. As shown in the example shown in FIG. 6, the time phase image 631 of the time phase 1 is set as the reference phase image.

A method such as optical flow can be used for calculation of the displacement amount. As a simple method, a difference in position information of feature points of an image or an ROI set in the image may be used as the deviation amount.

When the reference time phase image is the time phase image 631 of the time phase 1, the displacement correction unit 340 corrects the time phase images 632 to 63N using the displacement amount 640 thereof from the time phase image 631 (S5). Here, the displacement-corrected data created by correction is images obtained by correcting the displacement amount 640 of the time phase images 631 to 63N. Alternatively, the displacement-corrected data created by correction may be partial image data of time phase images 631 to 63N corresponding to the measurement data 610, obtained by performing the inverse FFT on each piece of the measurement data 610 used for generating the time phase images 631 to 63N and then correcting the displacement amount 640. Alternatively, the displacement-corrected data created by correction may be measurement data in which displacement is corrected corresponding to the measurement data 610, created by performing the inverse FFT on each piece of the measurement data 610 used for generating the time phase images 631 to 63N, correcting the displacement amount 640, and then performing FFT.

In the data related to each time phase image, the deviation amount due to the motion is corrected. However, the image generated with only the data related to each time phase image is an image reconstructed with a relatively small amount of data, and thus has a low SN ratio and a low image quality with image blurring. High image quality can be achieved by integrating all the measurement data 610 collected by the image integration unit 350. For this, when the displacement-corrected data is an image, the image integration unit 350 first performs FFT on each of the time phase images in which displacement from the time phase images 631 to 63N is corrected to return the time phase images to measurement space data (S61), integrates reconverted measurement space data 651 to 65N of each time phase by complex addition or the like (S62), and then uses integrated measurement space data 650 to perform image reconstruction (S63). Examples of a method for integrating the reconverted measurement space data 651 to 65N also include a method of using weighted complex addition, which uses weights related to the measurement data 621 to 62N of each time phase. Accordingly, it is possible to achieve high image quality of the integrated image by further reflecting actually-measured high-frequency data.

Similarly to the reconstruction performed by the multiphase image generation unit 320, the image reconstruction may be performed by the inverse FFT or the like, or may be performed by the compressed sensing. When the displacement-corrected data is measurement data corresponding to the measurement data 610 in which the displacement is corrected, processing of S61 is not necessary, whereas S62 and the subsequent processing are the same.

Regarding the integration of time phase images, instead of returning to the measurement space data and integrating the measurement space data, the images of each time phase corrected by the displacement correction unit 340 may be integrated by complex addition to generate the integrated image. Thus, the calculation can be simplified.

The integrated image is stored in the storage device 116 or displayed on the display 111 as necessary (S7).

According to the present embodiment, a sampling interval (an interval of sampling data of each line) of the measurement data including the low-frequency data and the high-frequency data is set shorter than the motion cycle so that the low-frequency data in the k-space has a sampling density higher than the sampling density of the high-frequency data. Further, the measurement data obtained by performing the radial scan in a predetermined scanning order is used to perform division, displacement correction, and integration of time phases. Thus, it is possible to obtain a high-quality image of a desired time phase in which the influence of the motion is reduced without requiring breath holding or extending the imaging time.

By changing a time phase image to be set as reference when the displacement amount is calculated to perform the displacement correction, it is possible to obtain a high-quality image for any time phase or all of time phases.

### First Modification

The first embodiment has described a case where the radial scan is adopted as a k-space scanning method, but for example, the spiral scan or the Cartesian scan in which k-space is scanned in parallel may also be adopted.

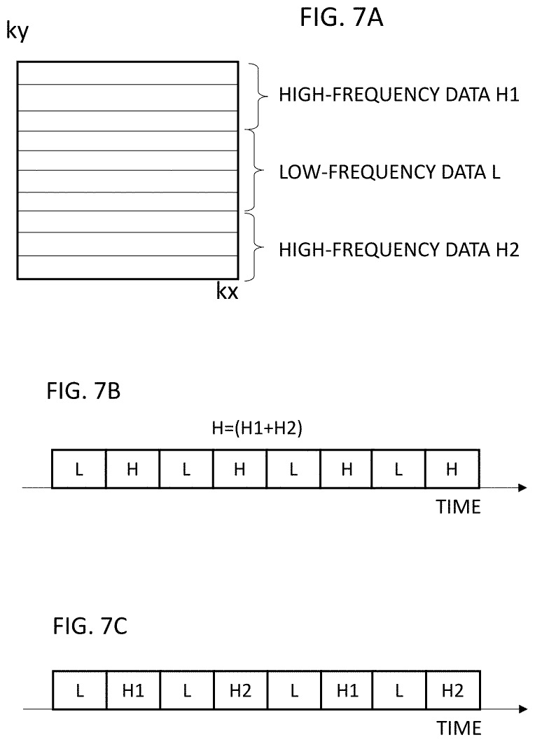

In a case of three-dimensional Cartesian scan as shown in FIG. 7A, for example, as shown in FIGS. 7B and 7C, it is possible to alternately perform scan on low-frequency data L near zero encoding of phase encoding and scan on high-frequency data H (H1, H2) on both sides of L and repeat the scan such that the number of scans of the low-frequency data L is larger than that of the high-frequency data H, so as to achieve sampling such that the low-frequency data L has a higher sampling density and the high-frequency data H has a lower sampling density. Regarding slice encoding steps, for example, low-frequency data of several encodings from the center of the k-space (zero encoding) may be sampled without skipping any encoding step. On the other hand, data of the other region (high-frequency data) may be collected by repeating sparse measurement (undersampling) while changing a collection line of the high-frequency data, until data completely fills the entire k-space over a plurality of times of k-space data collection.

By using one of these methods or combining both, sampling is performed in the three-dimensional k-space such that the low-frequency data has a higher sampling density and the high-frequency data has a lower sampling density.

Also in the present modification, by setting the sampling interval of collecting predetermined data to be sufficiently shorter than the motion cycle (e.g., by setting the sampling interval of collecting each set of low-frequency data L and high-frequency data H to be sufficiently shorter than the motion cycle), the measurement data of each time phase collected from the measurement data measured over a plurality of motion cycles includes low-frequency data and high-frequency data. Therefore, it is possible to use the measurement data of each time phase to obtain an image having a spatial resolution to an extent that enables calculation of the displacement amount.

Therefore, the reconstruction of the time phase image using the measurement data, the displacement amount calculation using each time phase image, the displacement correction of each time phase image, and integration of the corrected time phase images can be performed in the same manner as in the first embodiment.

### Second Embodiment

The first embodiment has described the case where the number of time phases of the motion cycle and the reference time phase for determining the displacement amount are set by default on a system side, but an MRI apparatus according to the present embodiment is characterized in that these conditions are designated by the user.

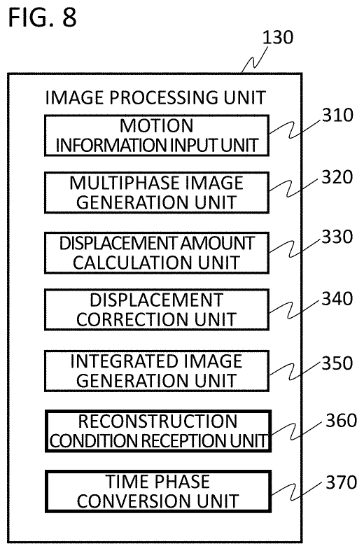

Hereinafter, the present embodiment will be described focusing on differences from the first embodiment. FIG. 8 shows a configuration of the image processing unit 130 according to the present embodiment. In FIG. 8, elements that perform the same processing as those in FIG. 2 are denoted by the same reference signs, and redundant description thereof will be omitted. As shown in FIG. 8, the image processing unit 130 according to the present embodiment is added with a reconstruction condition reception unit (reception unit) 360 and a time phase conversion unit 370.

The reconstruction condition reception unit 360 receives designation by the user, such as into how many time phases the motion cycle is to be divided, and which time phase image is to be formed and displayed. Reception of the designation by the user is performed by the control unit (display control unit) 140 causing the display 111 to display the GUI for receiving a condition and the user inputting a desired condition via the input device.

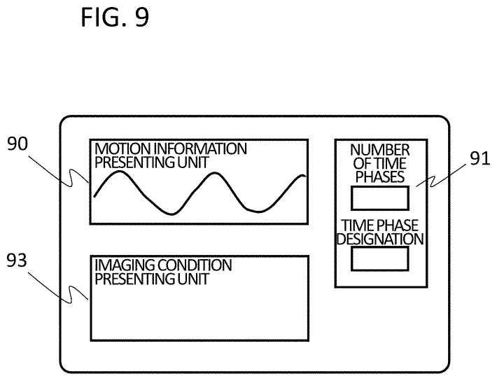

An example of the GUI displayed on the display is shown in FIG. 9. The example shown in FIG. 9 includes a motion display block 90 that displays motion information received from the motion detection device 20 by the motion information input unit 310, and a condition input block 91 for designating the number of time phases and a predetermined time phase. For example, the user views a graph showing a motion cycle displayed in the motion display block 90, checks a range (length of time) appropriate for division into time phases, and inputs the range as the number of time phases into the condition input block 91. Alternatively, a cursor or the like may be displayed in the motion display block 90, so that the range can be designated with the cursor. In this case, the number of time phases determined by the range designation is displayed in the condition input block 91.

The user may designate the time phase of the image to be reconstructed based on the graph indicating the motion cycle. For example, in a case of breathing, an exhale period, an inhale period, and the like can be designated, and in a case of heartbeat, a predetermined time period can be designated based on an R-wave. The designation method may be any method, such as designation by a pointer or the like on the graph, or assigning reference signs of 1 to N to the time phases and designating the number by the condition input block 91. Designation of a plurality of time phases may be received instead of one. In this case, the integrated image is reconstructed for the plurality of time phases.

A time period in which a change rate of the motion is equal to or less than a predetermined threshold may be designated. In this case, a GUI for receiving the threshold may be displayed, or a predetermined threshold may be set in advance according to a type of motion (cardiac cycle or respiratory cycle) to automatically create an image of a time phase of a motion stable period among the designated number of time phases by the user.

In the designation of the number of time phases, imaging conditions to be considered in the designation of the number of time phases, such as imaging parameters (e.g., TR) used for imaging, and optionally imaging regions, subject information, and the like may be displayed in a reference information display block 93.

Processing of the measurement unit 100 and the image processing unit 130 in the present embodiment is the same as that in the first embodiment or the modification thereof, except that the number of time phases and the time phase images to be displayed are designated by the user. A method for reconstructing a desired time phase image thereof may be either one of the two methods described below.

In one method, when the displacement amount calculation unit 330 calculates the displacement amount of the time phase image, the displacement amount calculation unit 330 uses the image of the time phase designated by the user as the reference time phase image to calculate the displacement amount of other time phase images, and the displacement correction unit 340 performs displacement correction on the data related to the time phase images. Then, the image integration unit 350 integrates the time phase images, and thus it is possible to obtain an integrated image of a time phase as desired by the user. In this case, the time phase conversion unit 370 is unnecessary.

In the other method, similarly to the first embodiment, the displacement amount calculation unit 330 uses the image of the time phase set by default as the reference time phase image and calculates the displacement amount of other time phase images, the displacement correction unit 340 performs the displacement correction on the data related to the time phase images, and the image integration unit 350 obtains the integrated image. This integrated image is subjected to displacement correction by the time phase conversion unit 370 using the displacement amount between the time phase image of the time phase designated by the user and the reference time phase image (displacement amount 640 calculated by the displacement amount calculation unit 330 in FIG. 6: S5), and is converted into an integrated image of the designated time phase.

Whichever method described above is adopted, either the method of image reconstruction after conversion into measurement space data for integration or the method of performing complex addition on the time phase images may be adopted.

According to the present embodiment, the user can select a time phase most suitable for diagnosis, and can be presented with information contributing to the diagnosis. In addition, instead of using one time phase as the reference time phase, by sequentially changing the time phase image as reference to repeat processing from displacement amount calculation and displacement correction to integrated image generation, images of a plurality of time phases can be generated. Thus, it is possible to acquire a time-series image with high image quality.

### Third Embodiment

The MRI apparatus of the present embodiment is characterized by having a function of generating a time-series image. The images of any or a plurality of time phases generated in the first and second embodiments are high-quality images obtained by integrating images after displacement correction. The present embodiment presents the time-series image, which has an image quality lower than that of the integrated image but enables tracking of time change. Hereinafter, the present embodiment will be described focusing on differences from the first and second embodiments.

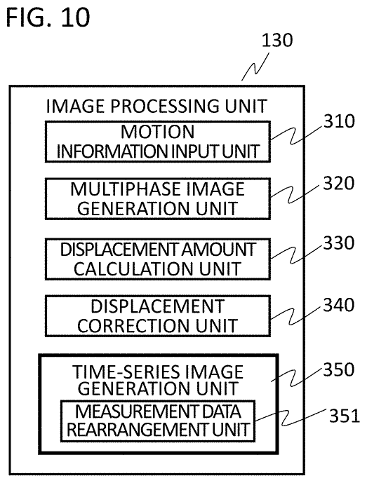

In the image processing unit 130 of the present embodiment, as shown in FIG. 10, the collection period reference image generation unit 350 of FIG. 2 is replaced with a time-series image generation unit 350 to describe the present embodiment. The time-series image generation unit 350 includes a measurement data rearrangement unit 351 that rearranges, in a data measurement order, data in which the displacement amount calculated by the displacement correction unit 340 related to the time phase image is corrected, and uses the rearranged measurement data to reconstruct the time phase image. Other elements are the same as those of the image processing unit 130 in the first embodiment. Hereinafter, processing of the image processing unit 130 in the present embodiment will be described focusing on processing of the time-series image generation unit 350.

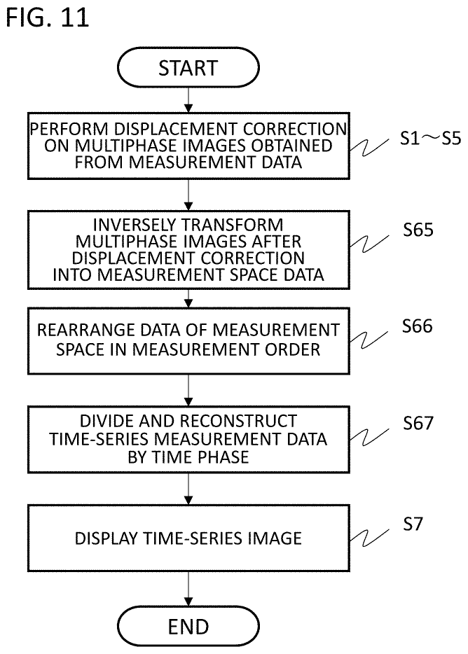

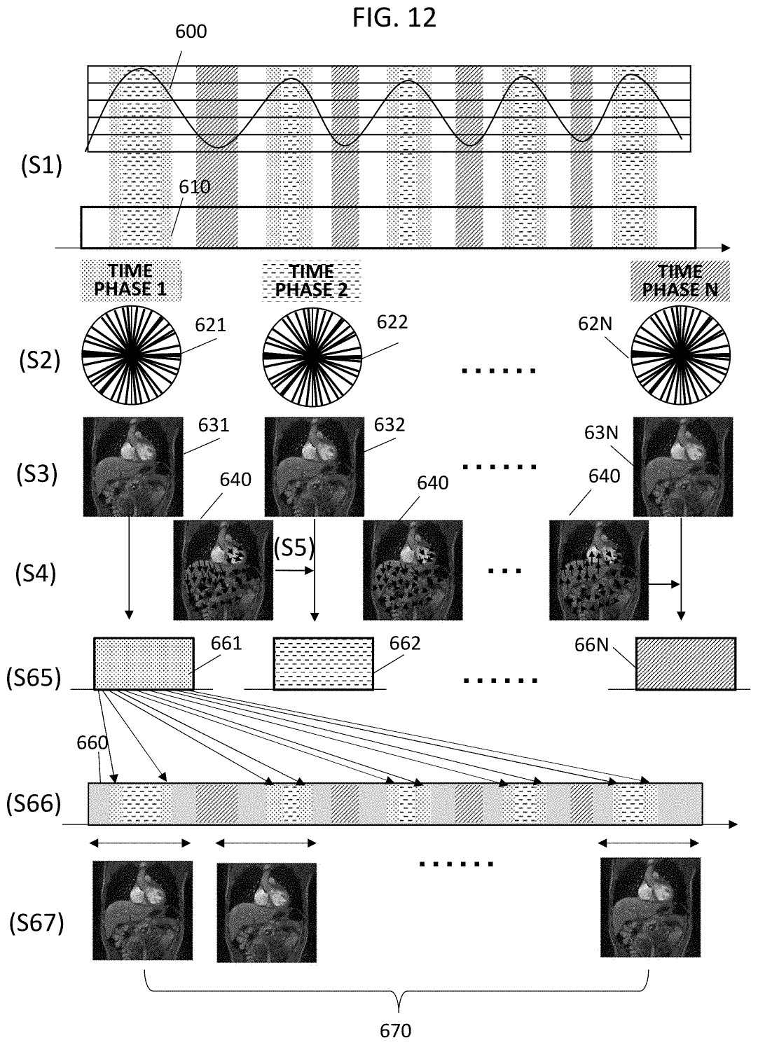

FIG. 11 shows a flow of processing performed by the image processing unit 130 of the present embodiment, and FIG. 12 shows contents of the processing. In FIG. 12, processing corresponding to steps shown in FIG. 11 are denoted by the same reference signs. The same elements as those in FIG. 6 are denoted by the same reference signs, and redundant description thereof will be omitted.

Time-series measurement data 610 obtained by repeating sampling in a plurality of cycles of motion is divided into measurement data 621 to 62N of each time phase (S1, S2), time phase images 631 to 63N are generated (S3), the displacement amount 640 between images is calculated using the time phase images for displacement correction (S4, S5), and displacement-corrected data 661 to 66N, in which displacement corresponding to the measurement data 610 related to the time phase images is corrected, is generated (S65). Here, the displacement-corrected data created by correction is partial data of the time phase images, obtained by performing the inverse FFT on the measurement data 610 used for generating the time phase images 631 to 63N, and correcting the displacement amount 640 for each piece of the measurement data 610. Alternatively, the displacement-corrected data is measurement data in which the calculated displacement is corrected, obtained by performing the FFT on the partial data. In either case, the displacement-corrected data is stored together with information at the time of acquiring the displacement-corrected data.

Next, the measurement data rearrangement unit 351 rearranges the converted measurement data (displacement-corrected data) 661 to 66N in the measurement order using the information at the time of acquiring the converted measurement data (S66). For example, in a case of the measurement data of the time phase 1, it is known which data is acquired at which timing, and thus, based on the information, each piece of data constituting the measurement data is arranged on a time axis to constitute time series data 660. Here, when original measurement data is measurement data of the Cartesian scan, the converted measurement data coincides in dimension with the original measurement data (one-dimensional sampling data), and thus may be directly rearranged in the measurement order. When the original measurement data is measurement data of the radial scan, the measurement data is converted into data arranged on the lattice of the k-space by gridding when forming the time phase image, and thus the measurement data 661 to 66N after the conversion becomes two-dimensional or three-dimensional array data. In this case, the measurement data may still be rearranged in the measurement order in the form of array data, or may be rearranged in the measurement order after being degridded to radial sampling data similar to the measurement data.

The time-series image generation unit 350 divides the time series data 660 obtained in this way by time phase of motion or at predetermined intervals, performs image reconstruction for each piece of divided measurement data, and generates a time-series image 670 (S67). At this time, the measurement data of a predetermined section of the time series data rearranged in the measurement order is arranged in the k-space based on a k-space scanning pattern at the time of imaging control of the measurement data 610, and is subjected to the image reconstruction by a method such as the inverse FFT or the compressed sensing. Alternatively, when the data rearranged in the measurement order is array data already arranged in the k-space, one piece of k-space array data is generated by performing processing such as complex addition on these data, and is subjected to the image reconstruction by a method such as the inverse FFT or the compressed sensing. Since the generated time-series image uses data of different time phases, it is possible to acquire a time-series image having higher image quality than that in the related art in a short time in a portion in motion. In addition, the time-series image becomes an image group in which the positional deviation of each time phase is eliminated and a morphological time change can be easily captured. The time-series image can be displayed with animation, for example (S7).

When the measurement data is divided by time phase, the image reconstruction may be performed by partially using overlapped measurement data between adjacent time phases. As a result, the image quality of the time-series image can be improved. Further, the rearranged measurement data may be divided by different time widths, instead of by a time phase the same as initially determined. For example, by dividing the measurement data at a time point when collection of data filling the entire k-space is completed and at time points when collection of high-density low-frequency data and low-density high-frequency data is completed, a time resolution of the time-series image decreases, but high image quality can be achieved.

Also in the present embodiment, similarly to the second embodiment, it is possible to receive user designation for the number of time phases and a time phase as a reference of displacement amount calculation via the reconstruction condition reception unit (reception unit) 360. As a result, it is possible to form and display a time-series image with reference to a position of the time phase designated by the user. The reconstruction condition designated by the user may be selectable between the spatial resolution or the time resolution of the time-series image. In this case, the time-series image generation unit 350 determines a size in a time direction of the time-series measurement data used for the time-series image based on the reconstruction condition received by the reconstruction condition reception unit 360, and increases the size in the time direction when the spatial resolution is prioritized and decreases the size in the time direction when the time resolution is prioritized.

According to the present embodiment, it is possible to present a time-series image in which the influence of the motion is reduced. Thus, it is possible to instantly present a state of change in position of a contrast agent in dynamic imaging or the like using contrast agent.

In the present embodiment, it is needless to say that the integrated image may be generated for a desired time phase or a plurality of time phases as in the first and second embodiments.

### Fourth Embodiment

The present embodiment is based on the third embodiment described above, further added with a function of presenting a change over time series in a region of interest, and is suitably applied to a case where a state in which a contrast agent reaches a target region is to be observed or an image at an optimal timing is to be obtained in contrast dynamic imaging or the like.

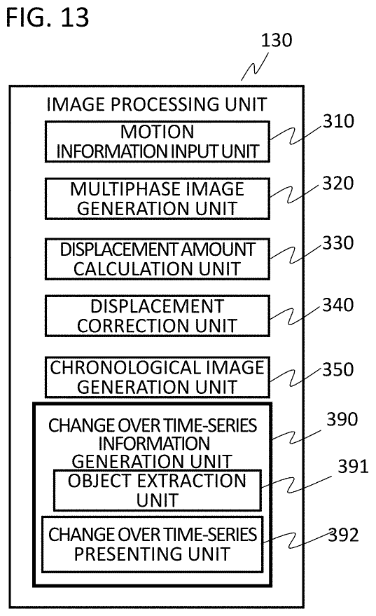

FIG. 13 shows an example of a functional block diagram of the image processing unit 130 according to the present embodiment. In FIG. 13, the same elements as those in FIGS. and 10 are denoted by the same reference signs, and differences from the above embodiments will be mainly described. As shown in FIG. 13, the image processing unit 130 includes a time change information generation unit 390 in addition to the configuration shown in FIG. 10. The time change information generation unit 390 includes an object extraction unit 391 and a change over time series presenting unit 392.

The time-series image generation unit 350 generates the time-series image based on the measurement data after displacement correction in the same manner as in the third embodiment. The object extraction unit 391 extracts a position of a specific tissue in the time-series image, for example, hepatic artery, portal vein, pancreas, and hepatic parenchyma. An extraction method of the specific tissue can be a general object extraction technique, i.e., extracting the specific tissue based on features of signal intensity and shape. Alternatively, when the specific tissue has a feature in shape, such as a linear shape or a shape having an apex, a specific line or a plurality of points may be extracted as the specific tissue rather than extracting the entire specific tissue.

Next, the change over time series presenting unit 392 calculates changes over time series in signal intensity and shape of the specific tissue extracted from the time-series images generated by the time-series image generation unit 350 (a change in inclination in a case of a line, and a change in distance between points in a case of points), and presents the changes over time series by, for example, displaying on a display.

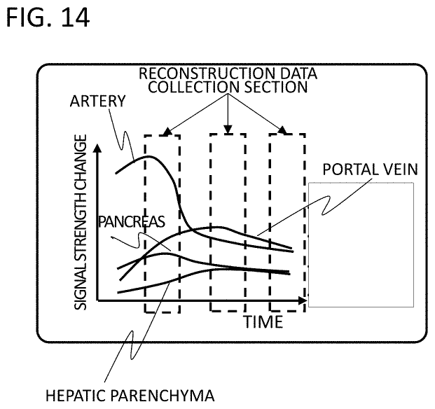

The change over time series presenting unit 392 may also display a GUI on the display by the reconstruction condition reception unit 360 while presenting time change information, and receive designation of a data collection section to be used for reconstruction by the user via the GUI. An example of such a GUI is shown in FIG. 14. In this example, signal intensity changes of the hepatic artery, the portal vein, the pancreas, and the hepatic parenchyma are shown in a graph, and data collection sections used for reconstruction can be specified by being surrounded by squares on this graph. Here, three sections are designated.

The time-series image generation unit 350 receives the user designation, and generates the time-series image based on the measurement data included in the section in the same manner as in the third embodiment. At this time, the reference time phase image among the plurality of time phase images may be a time phase image set by default, or may be an image of a time phase positioned at a center of the section. In addition, the reconstruction condition reception unit 360 may also receive the user designation for the time phase to be used as the reference.

Instead of designating the sections as shown in FIG. 14, a line may be received, or a point may be received with an arrow. The line or point designates one timing of change over time series, and the image integration unit 350 generates a time-series image of a predetermined section including the timing indicated by the line or point. Alternatively, it is also possible to select and present a time-series image at a timing designated from the time-series images already generated by the time-series image generation unit 350.

According to the present embodiment, it is possible to grasp, for example, a situation in which the contrast agent advances in blood based on signal intensity changes of a specific tissue, and to generate an image at an optimal timing.

What is claimed is:

1. A magnetic resonance imaging apparatus comprising:
a measurement unit configured to measure an echo signal generated by nuclear magnetic resonance from a subject and collect measurement data arranged in k-space;
an image processing unit configured to generate an image by processing the measurement data collected by the measurement unit; and
a control unit configured to control the measurement unit such that the measurement unit repeats sampling of measurement data including low-frequency data and high-frequency data such that the low-frequency data has a sampling density in the k-space higher than a sampling density of the high-frequency data, wherein
the image processing unit includes:
a motion information input unit configured to receive motion information of the subject;
a multiphase image generation unit configured to use the motion information to divide the measurement data into measurement data at a plurality segment of a motion cycle of the subject, and generate a multiphase image based on the divided measurement data;
a displacement amount calculation unit configured to calculate a displacement amount between the multiphase images due to the motion for the multiphase image;
a displacement correction unit configured to use the displacement amount to generate displacement-corrected data from data related to the each multiphase image; and
a collection period reference image generation unit configured to generate an image using the displacement-corrected data corresponding to the measurement data collected in any collection period by the measurement unit.

2. The magnetic resonance imaging apparatus according to claim 1, wherein
the measurement unit repeats the sampling in a cycle shorter than a motion cycle.

3. The magnetic resonance imaging apparatus according to claim 1, wherein
the measurement data measured by the measurement unit is three-dimensional k-space data including phase encoding in two directions, and
the sampling includes sampling of the low-frequency data and sampling of the high-frequency data of the three-dimensional k-space data.

4. The magnetic resonance imaging apparatus according to claim 1, wherein
the sampling is radial scan in which the k-space is radially sampled or spiral scan in which the k-space is spirally sampled.

5. The magnetic resonance imaging apparatus according to claim 1, wherein
the displacement amount calculation unit sets any time phase image among the multiphase images as a reference image, and calculates the displacement amount with respect to the reference image for a multiphase image other than the reference image,
the displacement correction unit uses the displacement amount to calculate the displacement-corrected data by converting data related to the multiphase image other than the reference image into data in which displacement is with reference to a time phase at which the reference image is acquired, and
the collection period reference image generation unit uses the displacement-corrected data corresponding to the measurement data collected in any collection period by the measurement unit to generate an image in which displacement is with reference to the time phase at which the reference image is acquired.

6. The magnetic resonance imaging apparatus according to claim 5, wherein
the displacement correction unit calculates, as the displacement-corrected data, multiphase displacement correction measurement data obtained by converting measurement data used for generating the multiphase image other than the reference image into measurement data in which the displacement amount is corrected, and
the collection period reference image generation unit generates the image in which displacement is with reference to the time phase at which the reference image is acquired, by performing image reconstruction processing such as inverse Fourier transform on the multiphase displacement correction measurement data corresponding to the measurement data collected in any collection period by the measurement unit.

7. The magnetic resonance imaging apparatus according to claim 5, wherein
the displacement correction unit calculates, as the displacement-corrected data, a multiphase displacement correction image obtained by converting the multiphase image other than the reference image into a time phase image in which displacement is with reference to the time phase at which the reference image is acquired, and
the collection period reference image generation unit generates the image in which displacement is with reference to the time phase at which the reference image is acquired by performing processing such as weighted complex addition on the multiphase displacement correction image corresponding to the measurement data collected in any collection period by the measurement unit.

8. The magnetic resonance imaging apparatus according to claim 1, wherein
the collection period reference image generation unit includes an image integration unit configured to integrate the multiphase image, and
the image integration unit generates an image using the displacement-corrected data corresponding to all the measurement data collected by the measurement unit, so as to generate an integrated image in which the multiphase images are integrated.

9. The magnetic resonance imaging apparatus according to claim 8, wherein
the displacement amount calculation unit repeats processing of setting any time phase image among the multiphase images as a reference image and calculating the displacement amount with respect to the reference image for a multiphase image other than the reference image, while changing the reference image,
the displacement correction unit uses a plurality of displacement amounts having different reference time phases to generate a plurality of pieces of displacement-corrected data from data related to the multiphase image, and
the image integration unit generates a plurality of integrated images with reference to different time phases.

10. The magnetic resonance imaging apparatus according to claim 1, further comprising:
a reception unit configured to receive a motion related condition related to a time phase of a motion cycle by a user, wherein
the multiphase image generation unit divides the measurement data in accordance with the condition received by the reception unit.

11. The magnetic resonance imaging apparatus according to claim 10, wherein
the motion related condition received by the reception unit includes the number of segmented the motion cycle, and
the multiphase image generation unit divides the measurement data based on the number of segmentation received by the reception unit.

12. The magnetic resonance imaging apparatus according to claim 10, wherein
the motion related condition received by the reception unit includes a desired time phase in the motion cycle, and
the displacement amount calculation unit sets the time phase received by the reception unit as a reference time phase, sets a multiphase image generated based on measurement data distributed to the reference time phase as a reference image, and calculates a displacement amount of the image other than the reference time phase with respect to the reference image.

13. The magnetic resonance imaging apparatus according to claim 1, wherein
the collection period reference image generation unit includes a time series image generation unit configured to generate a time-series image,
the time-series image generation unit includes a measurement data rearrangement unit configured to rearrange the displacement-corrected data corrected by the displacement correction unit in a measurement order and generate time-series measurement data in the measurement order, and
the time-series measurement data is used to generate the time-series image.

14. The magnetic resonance imaging apparatus according to claim 13, further comprising:
a reception unit configured to receive a reconstruction condition of the time-series image by a user.

15. The magnetic resonance imaging apparatus according to claim 14, wherein
the reconstruction condition includes one of a spatial resolution and a time resolution of the time-series image, and
the time-series image generation unit determines a size in a time direction of the time-series measurement data used for the time-series image based on the reconstruction condition received by the reception unit.

16. The magnetic resonance imaging apparatus according to claim 13, further comprising:
a tissue extraction unit configured to extract a desired portion or tissue based on the time-series image;
a change over time series calculation unit configured to calculate a change over time series in a signal value of the tissue extraction unit; and
a display control unit configured to cause a display unit to display the change over time series.

17. The magnetic resonance imaging apparatus according to claim 16, wherein
the display control unit causes the display unit to display a graphic user interface for receiving user designation for a section in which an image is to be reconstructed in the change over time series.

18. An image processing apparatus configured to process measurement data collected by repeating sampling of measurement data including low-frequency data and high-frequency data by magnetic resonance imaging such that the low-frequency data has a sampling density in the k-space higher than a sampling density of the high-frequency data, the image processing apparatus comprising:
an image information input unit configured to receive the measurement data and motion information of a subject when the measurement data is collected;
a multiphase image generation unit configured to use the motion information to divide the measurement data into measurement data at a plurality segment of a motion cycle, and generate a multiphase image of each time phase based on the divided measurement data;
a displacement amount calculation unit configured to calculate a displacement amount between the multiphase images due to the motion for the multiphase image;
a displacement correction unit configured to use the displacement amount to generate displacement-corrected data from data related to the each multiphase image; and
a collection period reference image generation unit configured to generate an image using the displacement-corrected data corresponding to the measurement data collected in any collection period by the measurement unit.
